(19) United States
(12) Patent Application Publication
(10) Pub. No.: US 2006/0217599 A1
(43) Pub. Date: Sep. 28, 2006

(54) Biological information collecting system

(75) Inventors: Yasunori Ohta, Kanagawa-ken (JP); Takeshi Kamiya, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/384,505

(22) Filed: Mar. 21, 2006

(30) Foreign Application Priority Data
Mar. 23, 2005 (JP) 2005-084610

(51) Int. Cl. A61B 5/00; G08B 1/08

(52) U.S. Cl. 600/300; 128/903; 340/539.23; 340/539.22

(57) ABSTRACT

A biological information collecting system has a biological information detecting sensor for measuring biological information of a biological subject and transmitting the biological information and measurement date/time data representing the measurement date/time, the biological information detecting sensor being disposed in a facility and/or mounted on the biological subject, an information storage device to be carried by the biological subject, and a managing device for detecting the biological subject as it enters or exits the facility, and acquiring and storing information from the information storage device. The information storage device with ID information of the biological subject being registered therein includes a transmitting and receiving unit for receiving the biological information and the measurement date/time data transmitted from the biological information detecting sensor, and transmitting the biological information and the measurement date/time data to the managing device, and a memory for storing the biological information and the measurement date/time data.

### BACKGROUND OF THE INVENTION

1. Field of the Invention:

The present invention relates to a biological information collecting system for measuring the biological information of a biological subject and managing the health condition of the biological subject based on the measured biological information.

2. Description of the Related Art:

Heretofore, there has been a known biological information collecting system as disclosed in Japanese Laid-Open Patent Publication No. 2000-126138. The conventional biological information collecting system has functions described below. A biological subject carries an IC card with an ID key inputted therein, and biological information measured by a biological information measuring means is temporarily recorded in the IC card.

The biological information recorded in the IC card is read together with the ID key and recorded in a biological information recording means. The biological information recording means stores the measured biological information as time-series data.

A wireless method using radio waves or the like may be employed for reading and writing of information from and to the IC card. Basically, the biological information measuring means may automatically be turned on to measure the biological information while the biological subject is not conscious of the measurement.

However, in the conventional system, it has not been disclosed that the system records biological information of individuals together with measurement date/time data information in an IC card. The conventional biological information collecting system is not based on a concept for storing in an IC card the biological information that is measured a plurality of times during a given time period, or storing different types of biological information together with their respective biological information codes in an IC card.

### SUMMARY OF THE INVENTION

It is an object of the present invention to provide a biological information collecting system for acquiring biological information from a biological subject while the biological subject is not conscious of the measurement (the acquirement), recording the biological information of the biological subject acquired during a given time period, together with measurement date/time data information, and providing an examiner such as an medical doctor with the acquired information collectively, thereby allowing the examiner to analyze the health condition etc of the biological subject highly accurately.

A biological information collecting system according to the present invention includes a biological information detecting sensor for measuring biological information of a biological subject and transmitting the biological information and measurement date/time data information representing a date/time at which the biological information is measured, the biological information detecting sensor being disposed in a facility and/or mounted on the biological subject, and an information storage device to be carried by the biological subject, the information storage device with ID information of the biological subject being registered therein comprising receiving means for receiving the biological information and the measurement date/time data information transmitted from the biological information detecting sensor, memory means for storing a plurality of pieces of the biological information measured during a given time period and a plurality of pieces of the measurement date/time data information, and transmitting means for transmitting the ID information, the biological information and the measurement date/time data information.

With the above arrangement, the biological information collecting system can acquire the biological information from a biological subject while the biological subject is not conscious of the measurement (the acquirement). The biological information collecting system can record the biological information of the biological subject acquired during a given time period, together with the measurement date/time data information to provide an examiner such as a medical doctor with the information collectively. The examiner can thus analyze the health condition etc of the biological subject highly accurately, based on the change of biological information over time.

The biological information detecting sensor may transmit, in addition to the biological information, a biological information code for specifying the biological information, the receiving means may receive the biological information code in addition to the biological information, and the memory means may store the biological information and the measurement date/time data information, in association with the biological information code.

The biological information collecting system may further include a managing device comprising entry/exit detecting means for detecting the biological subject when the biological subject enters or exits the facility, communicating means for communicating with the information storage device carried by the biological subject to acquire the information stored in the memory means when the entry/exit detecting means detects the biological subject exiting the facility, and information memory means for storing the information acquired by the communicating means.

The biological information collecting system according to the present invention, as described above, can acquire the biological information from a biological subject while the biological subject is not conscious of the measurement (the acquirement), and record the biological information of the biological subject acquired during a given time period, together with the measurement date/time data information to provide an examiner such as a medical doctor with the information collectively. The examiner can thus analyze the health condition etc of the biological subject highly accurately.

The above and other objects, features, and advantages of the present invention will become more apparent from the following description when taken in conjunction with the accompanying drawings in which preferred embodiments of the present invention are shown by way of illustrative example.

### BRIEF DESCRIPTION OF THE DRAWINGS

FIG. 1 is a block diagram of a biological information collecting system according to an embodiment of the present invention;

FIG. 2 is a block diagram of a biological information detecting sensor of the biological information collecting system according to the embodiment;

FIG. 3 is a plan view of a card-type information storage device;

FIG. 4 is a perspective view of a ring-type information storage device;

FIG. 5 is a perspective view of a pierce-type information storage device;

FIG. 6 is a block diagram of an information storage device of the biological information collecting system according to the embodiment;

FIG. 7 is a diagram showing an example of details of a biological information file;

FIG. 8 is a block diagram of a managing device of the biological information collecting system according to the embodiment;

FIG. 9 is a flowchart of a processing sequence of the biological information detecting sensor;

FIG. 10 is a flowchart of a processing sequence of the information storage device; and

FIG. 11 is a flowchart of a processing sequence of the managing device.

### DESCRIPTION OF THE PREFERRED EMBODIMENT

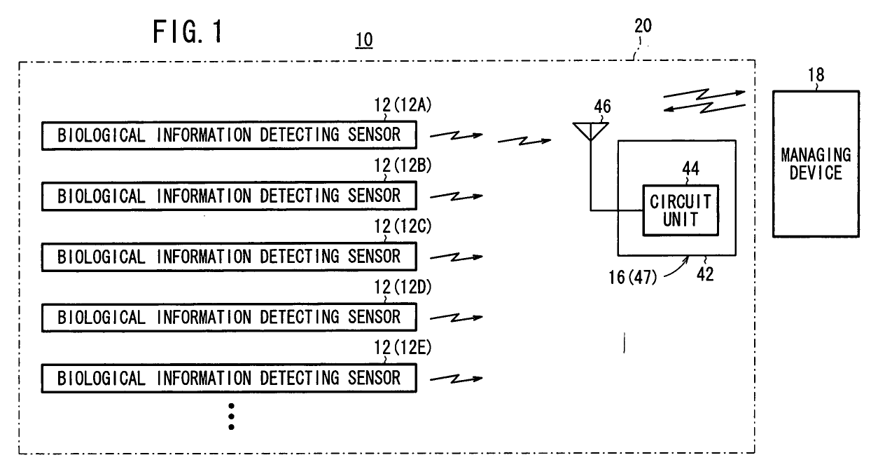

A biological information collecting system according to an embodiment of the present invention will be described below with reference to FIGS. 1 through 11.

As shown in FIG. 1, the biological information collecting system, generally designated by 10, has a plurality of biological information detecting sensors 12, an information storage device 16 that is carried by a biological subject (not shown), and a managing device 18.

The biological information detecting sensors 12 are disposed in a facility 20 that a biological subject enters and exits, and/or mounted on a biological subject. The biological information detecting sensors 12 can measure the biological information of the biological subject and send measurement date/time data information representing a date/time at which the biological information is measured, together with the measured biological information.

The biological information detecting sensors 12 may comprise an electronic clinical thermometer 12A, an electronic scale 12B, a electronic body fat meter 12C, an electronic blood pressure gage 12D, an electronic blood glucose level sensor 12E, etc.

The electronic clinical thermometer 12A measures the body temperature of the biological subject and sends information about the measurement date/time and the measured body temperature, i.e., a measured value, measurement date/time data, and a biological information code representing the body temperature. The electronic scale 12B measures the body weight of the biological subject and sends information about the measurement date/time and the measured body weight, i.e., a measured value, measurement date/time data, and a biological information code representing the body weight. The electronic body fat meter 12C measures the body fat of the biological subject and sends information about the measurement date/time and the measured body fat, i.e., a measured value, measurement date/time data, and a biological information code representing the body fat. The electronic blood pressure gage 12D measures the blood pressure of the biological subject and sends information about the measurement date/time and the measured blood pressure, i.e., a measured value, measurement date/time data, and a biological information code representing the blood pressure. The electronic blood glucose level sensor 12E measures the blood glucose level of the biological subject and sends information about the measurement date/time and the measured blood glucose level, i.e., a measured value, measurement date/time data, and a biological information code representing the blood glucose level.

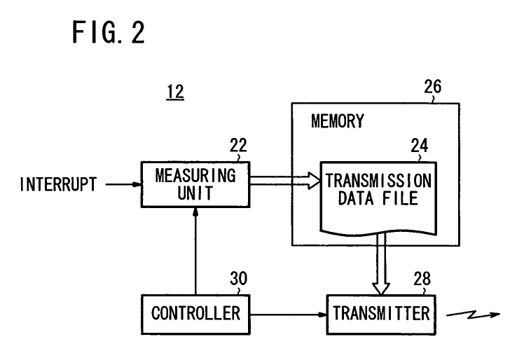

The biological information detecting sensors 12 may automatically be turned on periodically to measure necessary data. As shown in FIG. 2, each of the biological information detecting sensors 12 basically comprises a measuring unit 22 for measuring data based on a periodical internal interrupt, a memory 26 having a transmission data file 24 for temporarily storing data measured by the measuring unit 22 (biological information: measured value) and measurement date/time data, a transmitter 28 for sending the data (the biological information and the measurement date/time data) stored in the transmission data file 24 of the memory 26, together with the code specifying the biological information (biological information code), and a controller 30 for controlling the measuring unit 22 and the transmitter 28.

As shown in FIG. 1, the information storage device 16 carried by the biological subject has a wireless IC tag 47 (also referred to as an RFID tag or an electronic tag) in which a circuit unit 44 (including an IC chip or IC chips) and a small-size antenna 46 are mounted integrally. The circuit unit 44 is mounted in a package 42, and the small-size antenna is arranged in the package 42 and connected to the circuit unit 44.

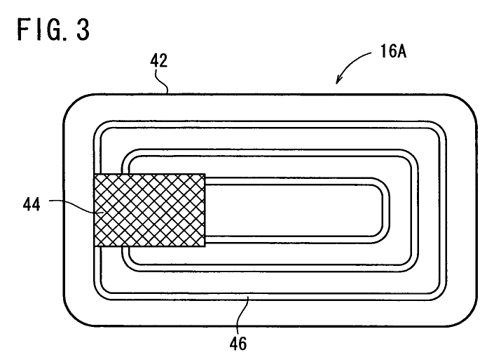

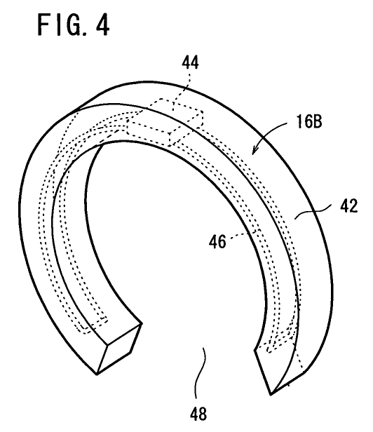

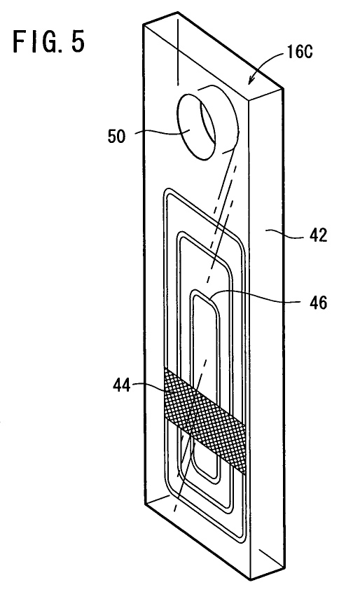

The information storage device 16 may be a card-type information storage device 16A (see FIG. 3), or a ring-type information storage device 16B (see FIG. 4), or a pierce-type information storage device 16C (see FIG. 5) that can easily be carried by the biological subject.

As shown in FIG. 3, the card-type information storage device 16A has a card-shaped package 42 made of synthetic resin and having the size and thickness as a credit card, for example. A circuit unit 44 and a small-size antenna 46 are mounted in the package 42.

As shown in FIG. 4, the ring-type information storage device 16B has a substantially ring-shaped package 42 made of synthetic resin. A circuit unit 44 and a small-size antenna 46 are mounted in the package 42. The package 42 has a cutout 48 defined partly therein to make it wearable on fingers of different diameters of various biological subjects. The package 42 can be put on a thick finger by widening the cutout 48 through elastic deformation or plastic deformation.

As shown in FIG. 5, the pierce-type information storage device 16C has a plate-like package 42 made of synthetic resin and having a through hole 50 defined therein for passage of a wire to be attached to an ear of the biological subject. A circuit unit 44 and a small-size antenna 46 are mounted in the package 42.

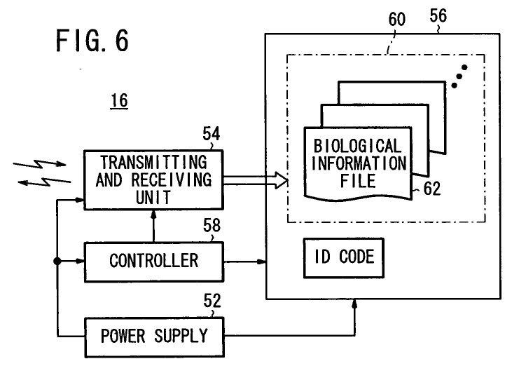

The information storage device 16 has a data transmitting and receiving function and an information storing function. As shown in FIG. 6, the information storage device 16 comprises a power supply 52, a transmitting and receiving unit 54, a memory 56, and a controller 58 for controlling the transmitting and receiving unit 54 and the memory 56.

The transmitting and receiving unit 54 receives information from the biological information detecting sensors 12, and an instruction from the managing device 18. Based on the instruction from the managing device 18, the transmitting and receiving unit 54 transmits information stored in the memory 56.

The memory 56 stores information that is received from the biological information detecting sensors 12 by the transmitting and receiving unit 54.

The memory 56 also stores ID information (ID codes) at certain addresses and also stores biological subject information 60.

The biological subject information 60 has a plurality of biological information files 62 corresponding to the respective types of biological information codes.

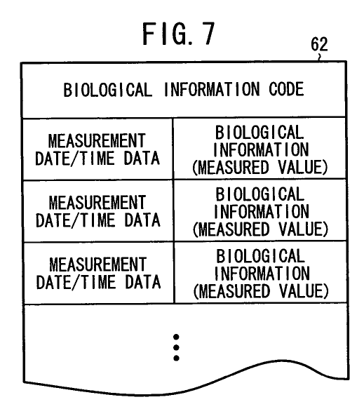

As shown in FIG. 7, each of the biological information files 62 contains measurement date/time data and biological information (measured values) at the measurement dates/times represented by the measurement date/time data, which are related to each other, in addition to the biological information code.

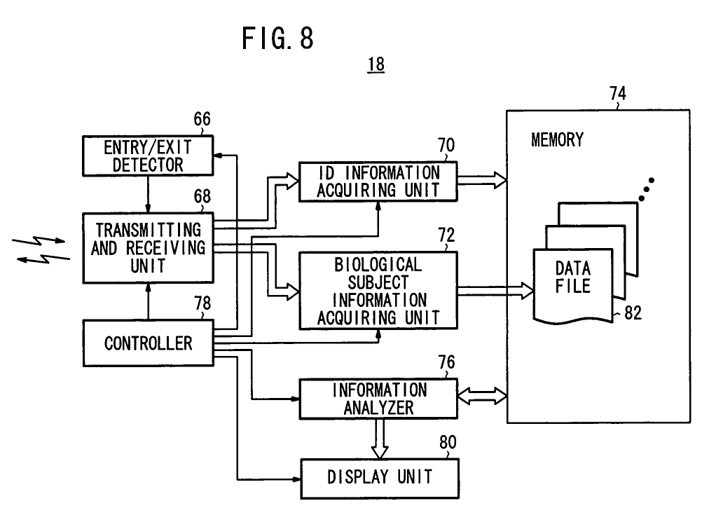

As shown in FIG. 8, the managing device 18 comprises an entry/exit detector 66, a transmitting and receiving unit 68, an ID information acquiring unit 70, a biological subject information acquiring unit 72, a memory 74, an information analyzer 76, and a controller 78 for controlling the entry/exit detector 66, the transmitting and receiving unit 68, the ID information acquiring unit 70, the biological subject information acquiring unit 72, the memory 74, and the information analyzer 76. To the managing device 18, there is connected a display unit 80 for displaying analytical results from the information analyzer 76, for example.

The entry/exit detector 66 is installed at an entrance/exit of the facility 20 for detecting the biological subject as it enters and exits the facility 20. The entry/exit detector 66 may be in the form of an optical sensor or the like.

When the entry/exit detector 66 detects the biological subject as it enters the facility 20, the ID information acquiring unit 70 instructs, through the transmitting and receiving unit 68, the information storage device 16 carried by the biological subject to send the ID code, and receives the ID code that is sent from the information storage device 16 based on the instruction.

When the entry/exit detector 66 detects the biological subject as it exits the facility 20, the biological subject information acquiring unit 72 instructs, through the transmitting and receiving unit 68, the information storage device 16 carried by the biological subject to send the biological subject information 60, and receives the biological subject information 60 that is sent from the information storage device 16 based on the instruction.

The memory 74 has a plurality of logically assigned data files 82 corresponding to the respective types of ID codes. Each of the data files 82 stores an ID code and the biological subject information 60 corresponding to the ID code, i.e., a plurality of biological information files 62 corresponding to the types of biological information codes.

Based on an instruction (including ID information) from the controller 78, the information analyzer 76 reads one of the data files 82 stored in the memory 74 which corresponds to the ID information from the controller 78, and performs a statistical process on data in the data file 82 to analyze the health condition of the biological subject corresponding to the ID code. The analytical result is displayed by the display unit 80.

The managing device 18 may be installed in a medical organization so that an examiner such as a medical doctor can make a professional data analysis with the information on the display unit. The biological subjects may also receive advice from the doctor.

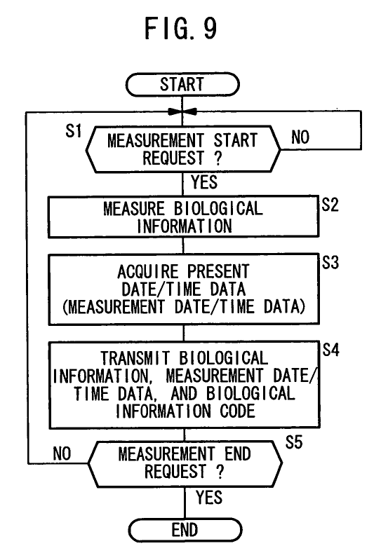

Operation of the biological information collecting system 10 according to the embodiment of the present invention will be described below with reference to FIGS. 9 through 11.

First, operation of each of the biological information detecting sensors 12 will be described below with reference to FIGS. 2 and 9. In step S1 shown in FIG. 9, it is determined whether there is a measurement start request or not based on whether there is a periodical internal interrupt..

If there is a measurement start request, then control goes to step S2 in which the measuring unit 22 (see FIG. 2) starts measuring data, such as the body temperature, the body weight, the body fat, the blood pressure, or the blood glucose level of the biological subject. The measured value (biological information) is stored in the transmission data file 24 of the memory 26.

When the data measurement is finished, control goes to step S3 in which the measuring unit 22 acquires present date/time data and stores the acquired present date/time data into the transmission data file 24. The measuring unit 22 can acquire the present date/time data easily from a clock (not shown) in the biological information collecting system 10.

Thereafter, in step S4, the transmitter 28 sends the data (the biological information and the measurement date/time data) stored in the transmission data file 24 of the memory 26, together with the biological information code. The data sent from the transmitter 28 is received by the information storage device 16 that is carried by the biological subject who is being currently measured.

In step S5, it is determined whether there is a measurement end request due to a power supply interruption or a maintenance interrupt for the biological information detecting sensor 12 or not. If there is no measurement end request, then control goes back to step S1 to wait for a next measurement cycle. If there is a measurement end request, then the processing sequence of the biological information detecting sensor 12 is put to an end.

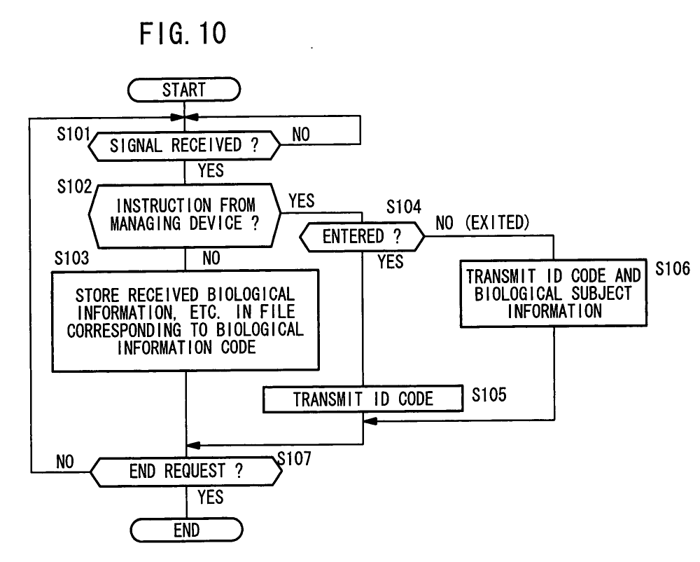

Operation of the information storage device 16 carried by the biological subject will be described below with reference to FIGS. 6 and 10.

In step S101 shown in FIG. 10, the transmitting and receiving unit 54 waits for a signal sent from the biological information detecting sensors 12, and the managing device 18.

If there is a signal, then control goes to step S102 in which it is determined whether the signal is an instruction from the managing device 18 or not.

If the signal is not an instruction from the managing device 18, then control goes to step S103 in which the transmitting and receiving unit 54 receives information (a biological information code, biological information, and measurement date/time data) from the biological information detecting sensors 12. The received information is stored in the memory 56. Specifically, the measurement date/time data and the biological information are related to each other, and stored in one of the biological information files 62 assigned in the memory 56. The one of the biological information files 62 corresponds to the biological information code in the received information.

If the signal is an instruction from the managing device 18 in step S102, then control goes to step S104 to determine whether the instruction is an instruction to send the ID code upon the entry of the biological subject into the facility 20 or not.

If the instruction is an instruction to send the ID code upon the entry of the biological subject into the facility 20, then control goes to step S105 in which the ID code that is stored in the allocated address in the memory 56 is transmitted through the transmitting and receiving unit 54 to the managing device 18.

If the instruction is an instruction to send the biological subject information upon the exit of the biological subject from the facility 20, then control goes to step S106 in which the ID code and the biological subject information (biological information files 62) stored in the memory 56 are transmitted through the transmitting and receiving unit 54 to the managing device 18.

After step S103 or step S105 or step S106, control goes to step S107 to determine whether there is an end request due to a power supply interruption, removal from the biological subject, or a maintenance interrupt for the information storage device 16 or not. If there is no end request, then control goes back to step S101 to wait for a next signal. If there is an end request, then the processing sequence of the information storage device 16 is put to an end.

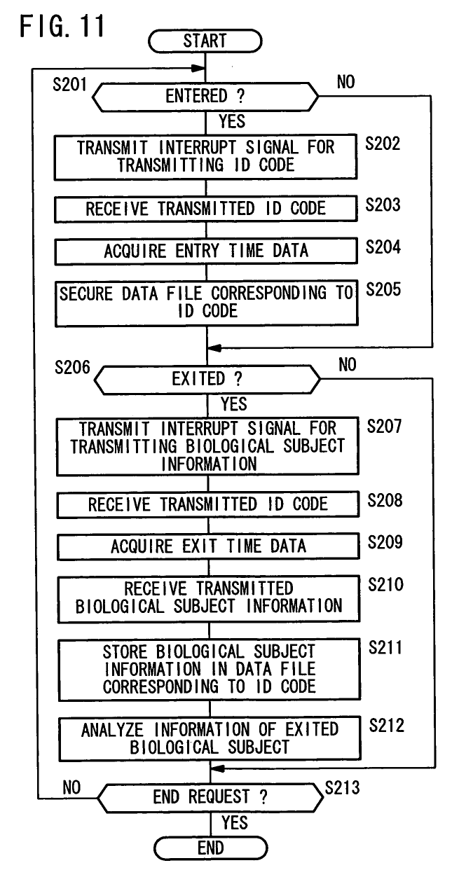

Operation of the managing device 18 will be described below with reference to FIGS. 8 and 11. In step S201 shown in FIG. 11, the entry/exit detector 66 determines whether a biological subject has entered the facility 20 or not. If the entry/exit detector 66 has detected the entry of a biological subject, then control goes to step S202 in which the transmitting and receiving unit 68 transmits an interrupt signal for instructing the information storage device 16 carried by the biological subject to transmit the ID code.

As described above, the instructed information storage device 16 transmits the ID code to the managing device 18 in step S105 shown in FIG. 10. In step S203, the ID information acquiring unit 70 of the managing device 18 receives the ID code transmitted from the information storage device 16 through the transmitting and receiving unit 68. In step S204, the ID information acquiring unit 70 acquires entry time data. The ID information acquiring unit 70 can acquire the entry time data easily from the clock (not shown) in the biological information collecting system 10.

In step S205, the data file 82 corresponding to the ID code is secured in the memory 74. The data file 82 has a storage capacity large enough to store the biological subject information 60 of the biological subject.

After step S205 or if the entry of a biological subject is not detected in step S201, control goes to step S206 in which the entry/exit detector 66 determines whether a biological subject has exited the facility 20 or not. If the entry/exit detector 66 has detected the exit of a biological subject, then control goes to step S207 in which the transmitting and receiving unit 68 transmits an interrupt signal for instructing the information storage device 16 carried by the biological subject to transmit the biological subject information 60.

As described above, the instructed information storage device 16 transmits the ID code and the biological subject information 60 to the managing device 18 in step S106 shown in FIG. 10. The transmitting and receiving unit 68 of the managing device 18 receives the ID code of the information transmitted from the information storage device 16 in step S208 and receives exit time data in step S209. In step S210, the biological subject information acquiring unit 72 acquires the biological subject information 60 of the information transmitted from the information storage device 16. In step S211, the biological subject information 60 is stored in one of the data files 82 which corresponds to the ID code.

In step S212, the information analyzer 76 reads the biological subject information 60 of the biological subject corresponding to the ID code, the entry time data, and the exit time data, and performs a statistical process on the biological subject information 60 to analyze the health condition of the biological subject corresponding to the ID code. The analytical results are displayed on the display unit 80.

In step S213, it is determined whether there is an end request due to a power supply interruption or a maintenance interrupt for the managing device 18 or not. If there is no end request, then control goes back to step S201 to repeat the processing from step S201. If there is an end request, then the processing sequence of the managing device 18 is put to an end.

The biological information collecting system 10 according to the embodiment of the present invention, as described above, can acquire the biological information from a biological subject and the measurement date/time data while the biological subject is not conscious of the measurement (the acquirement). The biological information collecting system 10 can record the biological information of the biological subject acquired during a given time period (for example, time period between the entry time and the exit time of the subject into and from the facility) together with the measurement date/time data to provide an examiner such as a medical doctor with the information collectively. Thus, the examiner can analyze the health condition etc of the biological subject highly accurately, based on the change of biological information over time.

In the above embodiment, the time period between the entry time and the exit time of a biological subject into and from the facility is assumed as a given time period. Alternatively, the given time period may be the time period between the oldest time and the latest time of the measurement times represented by the measurement time data recorded in the data buffer, or a constant time period of one hour, two hours, etc. It will be clear that the time period setting can thus be changed variously because the measurement time data are recorded in the data buffer.

Although a certain preferred embodiment of the present invention has been shown and described in detail, it should be understood that various changes and modifications may be made therein without departing from the scope of the appended claims.

What is claimed is:

1. A biological information collecting system comprising:
a biological information detecting sensor for measuring biological information of a biological subject and transmitting said biological information and measurement date/time data information representing a date/time at which said biological information is measured, said biological information detecting sensor being disposed in a facility and/or mounted on said biological subject; and
an information storage device to be carried by said biological subject;
said information storage device with ID information of said biological subject being registered therein comprising:
receiving means for receiving said biological information and said measurement date/time data information transmitted from said biological information detecting sensor;
memory means for storing a plurality of pieces of said biological information measured during a given time period and a plurality of pieces of said measurement date/time data information; and
transmitting means for transmitting said ID information, said biological information and said measurement date/time data information.

2. A biological information collecting system according to claim 1, wherein said biological information detecting sensor transmits, in addition to said biological information, a biological information code for specifying said biological information;
said receiving means receives said biological information code in addition to said biological information; and
said memory means stores said biological information and said measurement date/time data information, in association with said biological information code.

3. A biological information collecting system according to claim 1, further comprising a managing device,
said managing device comprising:
entry/exit detecting means for detecting said biological subject when said biological subject enters or exits said facility;
communicating means for communicating with said information storage device carried by said biological subject to acquire said information stored in said memory means when said entry/exit detecting means detects said biological subject exiting said facility; and
information memory means for storing said information acquired by said communicating means.
